(19) United States
(12) Patent Application Publication
(10) Pub. No.: US 2016/0025697 A1
(43) Pub. Date: Jan. 28, 2016

(54) METHODS FOR EVALUATING INSECT RESISTANCE IN A PLANT

(71) Applicants: PIONEER HI-BRED INTERNATIONAL, INC., Johnston, IA (US); E.I. DUPONT DE NEMOURS & COMPANY, Wilmington, DE (US)

(72) Inventors: Jessie L. Alt, Madrid, IA (US); Kevin A. Kriss, Wilmington, DE (US); Thomas Morris, Wilmington, DE (US); James K. Presnail, Saint Louis, MO (US); Molly Ryan-Mahmutagic, Waukee, IA (US)

(21) Appl. No.: 14/776,158

(22) PCT Filed: Mar. 10, 2014
(86) PCT No.: PCT/US14/22513

§ 371 Date: Sep. 14, 2015

Related U.S. Application Data
(60) Provisional application No. 61/788,016, filed on Mar. 15, 2013.

(51) Int. Cl. G01N 33/00

(52) U.S. Cl. CPC G01N 33/0098

(57) ABSTRACT

Methods are provided for evaluating the insect resistance of a plant of interest. Specifically, methods are provided for high-throughput screening of soybean plants for resistance to kudzu bugs and stink bugs, including the brown marmorated stink bug and the southern green stink bug. In some embodiments, infesting emergent soybean seedlings with second instar stink bug nymphs and maintaining the nymphs in a closed environment with the seedling for only about 7 days allows evaluation of the stink bug resistance of a soybean variety of interest. Also provided are insect resistant plants, particularly plants resistant to kudzu bugs and stink bugs, as well as seeds produced by the resistant plants. Plants disclosed herein can be used to transfer the resistant trait into plant lines of interest.

### FIELD OF THE INVENTION

The present invention relates to the field of insect resistance and pest management in crop plants.

### BACKGROUND OF THE INVENTION

Plant pests, including Hemipteran insect pests, are a major factor in the loss of the world's agricultural crops. Agriculturally significant Hemipteran insects include the southern green stink bug (Nezara viridula), brown marmorated stink bug (Halyomorpha halys) and kudzu bug (Megacopta cribraria). Stink bugs are phytophagous pentatomids, with a wide host range including plants with growing shoots and developing seeds or fruits. Currently, Hemipteran insect pests are generally controlled by chemical insecticides and crop rotation. The use of chemical insecticides, however, increases costs to farmers and can cause harmful effects on the ecosystem. Moreover, consumers and government regulators alike are becoming increasingly concerned with the environmental hazards associated with the production and use of synthetic agrochemicals.

Soybean (Glycine max) is an important and valuable field crop. The soybean is the world's leading source of vegetable oil and protein meal and is also used as a food source for both animals and humans. Soybean is widely used as a source of protein for animal feeds for poultry, swine, and cattle. Hemipteran insect pests, especially stink bugs, are one of the most damaging pests to soybean crops in the Southeastern United States, Argentina, and Brazil. Stink bugs primarily damage the soybean plant during the plant reproductive stages by piercing the pod and extracting nutrients, thereby damaging yield and quality of the crop.

Thus, in light of the significant impact of insect pests, particularly Hemipteran insect pests, on the yield and quality of soybean crops, a method of screening for insect resistant varieties of soybean is desirable. Existing methods for stink bug screening utilize field trials of mature plants or pod assays wherein a mature insect is offered soybean pods in order to determine resistance. However, when parts, such as pods, are removed from soybean plants, it is common for insect resistance to decrease during the time that the part is separated from the plant. Further, the time and space necessary for large scale screening in the field create a need for an effective alternative to field trials. Thus, methods for high-throughput screening of soybean plants for resistance to stink bugs are of particular interest.

### BRIEF SUMMARY OF THE INVENTION

Methods are provided for evaluating the insect resistance of a plant of interest. Specifically, methods are provided for high-throughput screening of soybean plants for resistance to kudzu bugs and stink bugs, including the brown marmorated stink bug and the southern green stink bug. In some embodiments, infesting emergent soybean seedlings with second instar stink bug nymphs and maintaining the nymphs in a closed environment with the seedling for only about 7 days allows evaluation of the stink bug resistance of a soybean variety of interest. Also provided are insect resistant plants, particularly plants resistant to kudzu bugs and stink bugs, as well as seeds produced by the resistant plants. Plants disclosed herein can be used to transfer the resistant trait into plant lines of interest.

### DETAILED DESCRIPTION

Many modifications and other embodiments of the inventions set forth herein will come to mind to one skilled in the art to which these inventions pertain having the benefit of the teachings presented in the foregoing descriptions and the associated drawings. Therefore, it is to be understood that the inventions are not to be limited to the specific embodiments disclosed and that modifications and other embodiments are intended to be included within the scope of the appended claims. Although specific terms are employed herein, they are used in a generic and descriptive sense only and not for purposes of limitation.

### I. Overview

The methods described herein are useful for evaluating the level of insect resistance in plants. As used herein, the term “insect resistance” refers to the ability of a plant to grow and develop in the presence of an insect pest. The term “plant pest” or “insect pest” refers to any organism that can cause harm to a plant by inhibiting or slowing the growth of a plant, by damaging the tissues of a plant, by weakening the immune system of a plant, reducing the resistance of a plant to abiotic stresses, and/or by causing the premature death of the plant. Insect resistance of a plant can be evaluated by comparing the insect resistance of a plant of interest to the insect resistance of a control plant known to be resistant to an insect pest or to a test plant known to be sensitive (i.e.

not resistant) to a particular insect pest. The methods described herein are particularly useful for rapidly screening large numbers of plants for insect resistance in a laboratory setting, without the necessity of field trials.

### II. Insect Pests

Methods disclosed herein are useful for evaluating the ability of plants to resist insect pests. Insect pests include any insect which has a larval or nymphal stage of development, including but not limited to insects selected from the orders Coleoptera, Diptera, Hymenoptera, Lepidoptera, Mallophaga, Homoptera, Hemiptera, Orthoptera, Thysanoptera, Dermaptera, Isoptera, Anoplura, Siphonaptera, Trichoptera, etc., particularly Hemiptera. In some embodiments, insect pests include Pentatomidae insects. As used herein, the term “insect” refers to an insect at any stage of development, including an insect nymph and an adult insect.

Insect pests for major crops include: Soybean: Aphis glycines, Soybean aphid; Euschistus (E. biformis, E. integer, E. quadrator, E. servus, E. tristigma), Brown stinkbug; Piezodorus guildinii, red banded stink bug; Pseudoplusia includens, soybean looper; Anticarsia gemmatalis, velvetbean caterpillar; Plathypena scabra, green cloverworm; Ostrinia nubilalis, European corn borer; Agrotis ipsilon, black cutworm; Spodoptera exigua, beet armyworm; Heliothis virescens, cotton budworm; Helicoverpa zea, cotton bollworm; Epilachna varivestis, Mexican bean beetle; Myzus persicae, green peach aphid; Empoasca fabae, potato leafhopper; Acrosternum hilare, green stink bug; Nezara viridula, Southern green stink bug; Halyomorpha halys, brown marmorated stink bug; Megacopta cribraria, Kudzu bug; Melanoplus femurrubrum, redlegged grasshopper; Melanoplus differentialis, differential grasshopper; Hylemya platura, seedcorn maggot; Sericothrips variabilis, soybean thrips; Thrips tabaci, onion thrips; Tetranychus turkestani, strawberry spider mite; Tetranychus urticae, twospotted spider mite; Maize: Halyomorpha halys, brown marmorated stink bug; Euschistus (E. biformis, E. integer, E. quadrator, E. servus, E. tristigma), brown stinkbug, Acrosternum hilare, green stink bug; Nezara viridula, Southern green stink bug; Bagrada hilaris, Bagrada bug; Ostrinia nubilalis, European corn borer; Agrotis ipsilon, black cutworm; Helicoverpa zea, corn earworm; Spodoptera frugiperda, fall armyworm; Diatraea grandiosella, southwestern corn borer; Elasmopalpus lignosellus, lesser cornstalk borer; Diatraea saccharalis, surgarcane borer; Diabrotica virgifera, western corn rootworm; Diabrotica longicornis barberi, northern corn rootworm; Diabrotica undecimpunctata howardi, southern corn rootworm; pest species in the family Elateridae, including species of the genera Aeolus, Agriotes, Conoderus, Hemicrepidus, and Limonius; Melanotus spp., wireworms; Cyclocephala borealis, northern masked chafer (white grub); Cyclocephala immaculata, southern masked chafer (white grub); Popillia japonica, Japanese beetle; Chaetocnema pulicaria, corn flea beetle; Sphenophorus maidis, maize billbug; Rhopalosiphum maidis, corn leaf aphid; Anuraphis maidiradicis, corn root aphid; Blissus leucopterus leucopterus, chinch bug; Melanoplus femurrubrum, redlegged grasshopper; Melanoplus sanguinipes, migratory grasshopper; Hylemya platura, seedcorn maggot; Agromyza parvicornis, corn blot leafminer; Anaphothrips obscrurus, grass thrips; Solenopsis milesta, thief ant; Tetranychus urticae, twospotted spider mite; Sorghum: Chilo partellus, sorghum borer; Spodoptera frugiperda, fall armyworm; Helicoverpa zea, corn earworm; Elasmopalpus lignosellus, lesser cornstalk borer; Feltia subterranea, granulate cutworm; Phyllophaga crinita, white grub; Eleodes, Conoderus, and Aeolus spp., wireworms; Oulema melanopus, cereal leaf beetle; Chaetocnema pulicaria, corn flea beetle; Sphenophorus maidis, maize billbug; Rhopalosiphum maidis; corn leaf aphid; Sipha flava, yellow sugarcane aphid; Blissus leucopterus leucopterus, chinch bug; Contarinia sorghicola, sorghum midge; Tetranychus cinnabarinus, carmine spider mite; Tetranychus urticae, twospotted spider mite; Wheat: Eurygaster integriceps, Sunn pest; Diuraphis noxia, Russian wheat aphid; Pseudaletia unipunctata, army worm; Spodoptera frugiperda, fall armyworm; Elasmopalpus lignosellus, lesser cornstalk borer; Agrotis orthogonia, western cutworm; Elasmopalpus lignosellus, lesser cornstalk borer; Oulema melanopus, cereal leaf beetle; Hypera punctata, clover leaf weevil; Diabrotica undecimpunctata howardi, southern corn rootworm; Russian wheat aphid; Schizaphis graminum, greenbug; Macrosiphum avenae, English grain aphid; Melanoplus femurrubrum, redlegged grasshopper; Melanoplus differentialis, differential grasshopper; Melanoplus sanguinipes, migratory grasshopper; Mayetiola destructor, Hessian fly; Sitodiplosis mosellana, wheat midge; Meromyza americana, wheat stem maggot; Hylemya coarctate, wheat bulb fly; Frankliniella fusca, tobacco thrips; Cephus cinctus, wheat stem sawfly; Aceria tulipae, wheat curl mite; Sunflower: Suleima helianthana, sunflower bud moth; Homoeosoma electellum, sunflower moth; zygogramma exclamationis, sunflower beetle; Bothyrus gibbosus, carrot beetle; Neolasioptera murtfeldtiana, sunflower seed midge; Cotton: Heliothis virescens, cotton budworm; Helicoverpa zea, cotton bollworm; Spodoptera exigua, beet armyworm; Pectinophora gossypiella, pink bollworm; Anthonomus grandis, boll weevil; Aphis gossypii, cotton aphid; Pseudatomoscelis seriatus, cotton fleahopper; Trialeurodes abutilonea, bandedwinged whitefly; Lygus lineolaris, tarnished plant bug; Melanoplus femurrubrum, redlegged grasshopper; Melanoplus differentialis, differential grasshopper; Thrips tabaci, onion thrips; Franklinkiella fusca, tobacco thrips; Tetranychus cinnabarinus, carmine spider mite; Tetranychus urticae, twospotted spider mite; Rice: Diatraea saccharalis, sugarcane borer; Spodoptera frugiperda, fall armyworm; Helicoverpa zea, corn earworm; Colaspis brunnea, grape colaspis; Lissorhoptrus oryzophilus, rice water weevil; Sitophilus oryzae, rice weevil; Nephotettix nigropictus, rice leafhopper; Blissus leucopterus leucopterus, chinch bug; Acrosternum hilare, green stink bug; Barley: Ostrinia nubilalis, European corn borer; Agrotis ipsilon, black cutworm; Schizaphis graminum, greenbug; Blissus leucopterus leucopterus, chinch bug; Acrosternum hilare, green stink bug; Euschistus servus, brown stink bug; Delia platura, seedcorn maggot; Mayetiola destructor, Hessian fly; Petrobia latens, brown wheat mite; Oil Seed Rape: Brevicoryne brassicae, cabbage aphid; Phyllotreta cruciferae, Flea beetle; Mamestra Configurata, Bertha armyworm; Plutella xylostella, Diamond-back moth; Delia ssp., Root maggots.

As used herein “Pentatomidae insects” is used to refer to any member of the Pentatomidae family. Accordingly, the methods disclosed herein are also useful in evaluating plant resistance to any Pentatomidae insect including representative genera and species such as, but not limited to, Acrocorisellus (A. serraticollis), Acrosternum (A. adelpha, A. hilare, A. herbidum, A. scutellatum), Agonoscelis (A. nubila), Alcaeorrhynchus (A. grandis, A. phymatophorus), Amaurochrous (A. brevitylus), Apateticus (A. anatarius, A. bracteatus, A. cynicus, A. lineolatus, A. marginiventris), Apoecilus, Arma (A. custos), Arvelius, Bagrada, Banasa (B. calva, B. dimiata, B. grisea, B. induta, B. sordida), Brochymena (B. affinis, B. cariosa, B. haedula, B. hoppingi, B. sulcata), Carbula (C. obtusangula, C. sinica), Chinavia, Chlorochroa (C. belfragii, C. kanei, C. norlandi, C. senilis, C. viridicata), Chlorocoris (C. distinctus, C. flaviviridis, C. hebetatus, C. subrugosus, C. tau), Codophila (C. remota, C. sulcata, C. varius), Coenus (C. delius, C. inermis, C. tarsalis), Cosmopepla (C. bimaculata, C. binotata, C. carnifex, C. decorata, C. intergressus), Dalpada (D. oculata), Dendrocoris (D. arizonesis, D. fruticicola, D. humeralis, D. parapini, D. reticulatus), Dolycoris (D. baccarum (sloe bug)), Dybowskyia (D. reticulata), Edessa, Erthesina (E. fullo), Eurydema (E. dominulus, E. gebleri (shield bug), E. pulchra, E. rugosa), Euschistus (E. biformis, E. integer, E. quadrator, E. servus, E. tristigma), Euthyrhynchus (E. floridanus, E. macronemis), Gonopsis (G. coccinea), Graphosoma (G. lineatum (stinkbug), G. rubrolineatum), Halyomorpha (H. halys (brown marmorated stinkbug)), Halys (H. sindillus, H. sulcatus), Holcostethus (H. abbreviatus, H. fulvipes, H. limbolarius, H. piceus, H. sphacelatus), Homalogonia (H. obtusa), Hymenarcys (H. aequalis, H. crassa, H. nervosa, H. perpuncata, H. reticulata), Lelia (L. decempunctata), Lineostethus, Loxa (L. flavicollis, L. viridis), Mecidea (M indicia, M major, M minor), Megarrhamphus (M. hastatus), Menecles (M. insertus, M. portacrus), Mormidea (M cubrosa, M. lugens, M pama, M pictiventris, M. ypsilon), Moromorpha (M tetra), Murgantia (M. angularis, M. tessellata, M varicolor, M. violascens), Neottiglossa (N. californica, N. cavifrons, N. coronaciliata, N. sulcifrons, N. undata), Nezara (N. smaragdulus, N. viridula (southern green stinkbug)), Oebalus (O. grisescens, O. insularis, O. mexicanus, O. pugnax, O. typhoeus), Oechalia (O. schellenbergii (spined predatory shield bug)), Okeanos (O. quelpartensis), Oplomus (O. catena, O. dichrous, O. tripustulatus), Palomena (P. prasina (green shield bug)), Parabrochymena, Pentatoma (P. angulata, P. illuminata, P. japonica, P. kunmingensis, P. metallifera, P. parataibaiensis, P. rufipes, P. semiannulata, P. viridicornuta), Perillus (P. bioculatus, P. confluens, P. strigipes), Picromerus (P. griseus), Piezodorus (P. degeeri, P. guildinii, P. lituratus (gorse shield bug)), Pinthaeus (P. humeralis), Plautia (P. crossota, P. stali (brown-winged green bug)), Podisus (P. maculiventris), Priassus (P. testaceus), Prionosoma, Proxys (P. albopunctulatus, P. punctulatus, P. victor), Rhaphigaster (R. nebulosa), Scotinophara (S. horvathi), Stiretrus (S. anchorago, S. fimbriatus), Thyanta (T. accerra, T. calceata, T. casta, T. perditor, T. pseudocasta), Trichopepla (T. aurora, T. dubia, T. pilipes, T. semivittata, T. vandykei), Tylospilus, and Zicrona.

In specific embodiments, methods disclosed herein are useful for evaluating and identifying plants that show resistance to Nezara viridula (Southern green stink bug), Halyomorpha halys (brown marmorated stink bug), Megacopta cribraria (Kudzu bug), Acrostemum hilare (green stink bug), Oebalus pugnax, (rice stink bug), Pentatoma rufipes (forest bug), Rhaphigaster nebulosa, and Troilus luridus.

Insects employed in the method disclosed herein can be used at any stage of development wherein the insect is capable of utilizing plant material as a food source. For example, insects can be used after the first instar, during the second instar, third instar, fourth instar, fifth instar, or any other developmental or adult growth stage. As used herein, the term “instar” is used to denote the developmental stage of the larval or nymphal forms of insects. As the insect proceeds through the developmental stages, the type of plant material available as a food source may change. For example, during the first instar, a nymph or larvae may not be able to use any plant material as a food source, including plant cotyledon or soybean pod. However, as the nymph progresses into the second instar, the ability to utilize plant material as a food source develops. Thus, in the second instar, a nymph may be able to use the cotyledon of a plant, but not the soybean pod. As the nymph matures beyond the second instar, it concurrently begins to acquire the ability to utilize more developed plant parts as a food source, such as the leaves or soybean pods. In fact, all plant parts are likely to be fed upon, but growing shoots and developing fruit are preferred. In specific embodiments, brown marmorated stink bugs or southern green stink bugs are selected at the second instar stage for use in the method disclosed herein. In other embodiments, kudzu bugs are selected at the fourth instar stage for use in the method disclosed herein.

In addition to advancing food sources throughout development, the time that an insect nymph can survive without food also changes as the nymph matures. For example, at the second instar stage, a nymph can survive on water without any food source for at least about 5-15 days, 6-12 days, 7-10 days, or 8-9 days. Specifically, a second instar nymph can survive on water without any food source for about 5 days, 6 days, 7 days, 8 days, 9 days, 10 days, 11 days, 12 days, 13 days, 14 days, or 15 days. Similarly, at the fourth or fifth instar stage, a nymph can survive on water without any food source for at least about 8-21 days, 10-18 days, 12-14 days, or 12-16 days. Specifically, a fourth instar nymph can survive on water without any food source for 8 days, 9 days, 10 days, 11 days, 12 days, 13 days, 14 days, 15 days, 16 days, 17 days, 18 days, 19 days, 20 days, or 21 days. The time in which an insect nymph can survive on water, without any food source can vary based on environmental conditions such as a temperature, humidity, and light, among others.

For example, brown marmorated stink bugs and southern green stink bugs at the second instar stage can survive for about 7 days without a food source and kudzu bugs at the fourth instar stage can survive for about 14 days without a food source. Thus, in certain embodiments, brown marmorated stink bugs or southern green stink bugs are selected at the second instar stage, infested onto a soybean seedling at the emergence stage and maintained in an enclosed environment for at least about 7 days. In other embodiments, kudzu bugs are selected at the fourth instar stage, infested onto a soybean seedling at the VC or V1 stage, and maintained in an enclosed environment for at least about 14 days.

### III. Plants

As used herein, the term plant includes plant cells, plant protoplasts, plant cell tissue cultures from which a plant can be regenerated, plant calli, plant clumps, and plant cells that are intact in plants or parts of plants such as embryos, endosperm, pollen, ovules, seeds, cotyledons, meristem, cotyledonary nodes, leaves, flowers, branches, fruit, kernels, ears, pods, cobs, husks, stalks, roots, root tips, anthers, grain and the like. The method disclosed herein may be used to evaluate the insect resistance of any plant species, including but not limited to monocots and dicots.

Examples of plant species of interest include, but are not limited to, corn (Zea mays), Brassica sp. (e.g., B. napus, B. rapa, B. juncea), alfalfa (Medicago sativa), rice (Oryza sativa), rye (Secale cereale), sorghum (Sorghum bicolor, Sorghum vulgare), millet (e.g., pearl millet (Pennisetum glaucum), proso millet (Panicum miliaceum), foxtail millet (Setaria italica), finger millet (Eleusine coracana)), sunflower (Helianthus annuus), safflower (Carthamus tinctorius), wheat (Triticum aestivum), soybean (Glycine max), tobacco (Nicotiana tabacum), potato (Solanum tuberosum), peanuts (Arachis hypogaea), cotton (Gossypium barbadense, Gossypium hirsutum), sweet potato (Ipomoea batatus), cassava (Manihot esculenta), coffee (Coffea spp.), coconut (Cocos nucifera), pineapple (Ananas comosus), citrus trees (Citrus spp.), peach (Prunus persica), cocoa (Theobroma cacao), tea (Camellia sinensis), banana (Musa spp.), avocado (Persea americana), fig (Ficus casica), guava (Psidium guajava), mango (Mangifera indica), olive (Olea europaea), papaya (Carica papaya), cashew (Anacardium occidentale), macadamia (Macadamia integrifolia), almond (Prunus amygdalus), sugar beets (Beta vulgaris), sugarcane (Saccharum spp.), oats, barley, vegetables, ornamentals, grasses and conifers.

As used in the method disclosed herein, plants can be infested at any stage during the life cycle of the plant. For example, plants can be infested at germination, emergence, growth, flowering, or pod production. Germination refers to the growth stage as the seed separates and the primary root pushes into the soil. Emergence signifies when the seedling pushes out of the soil, rooted by the primary root and a developing network of secondary roots. At the emergence stage, the stalk of the seedling, referred to as the hypocotyl, pulls buds, called cotyledons, up and out of the soil. During the growth stage, the hypocotyl straightens out, buds begin to emerge and become new leaves. At the flowering stage, the plant has achieved sufficient growth such that flowers appear on nodes of the stems. Finally, for plants producing pods, such as soybean plants, the plant enters the pod production stage, wherein the plant begins growing pods.

In certain embodiments soybean seeds are planted in the method disclosed herein. About 7 days after planting, they elongation of hypocotyl brings the cotyledons out of the soil, which starts the soybean emergence. This emergence stage is referred to as the VE stage. After emergence, the plant proceeds to the VC stage, where unifoliate leaves unroll in addition to cotyledons (one node). At the V1 stage, the soybean seedling has one unrolled trifoliate leaf, and two nodes. Subsequently, at the V2 stage, the seedling exhibits two unrolled trifoliate leaves, with three nodes. Thus, as used herein, soybean seedlings can be infested at the VE stage, VC stage, V1 stage, or V2 stage. As used herein, the “vegetative stage” of plant development includes the VE stage, VC stage, V1 stage, and V2 stage. In specific embodiments, soybean seedlings are infested with insects of interest at the VE stage or the VC stage.

Plants used in the method disclosed here can be evaluated for insect resistance that is native or transgenic. Native insect resistance refers to insect resistance that is not the result of transgenic procedures such as plant transformation. Alternatively, transgenic plant resistance can result from the introduction of heterologous polynucleotide or polypeptide sequence. As used herein, “heterologous” in reference to a polynucleotide or polypeptide sequence is a sequence that originates from a foreign species, or, if from the same species, is substantially modified from its native form in composition and/or genomic locus by deliberate human intervention.

### IV. Method of Evaluating Insect Resistance

The methods described herein provide a means for evaluating insect resistance of a plant. Specifically, the methods can be used as a high-throughput approach for screening plants for insect resistance or insecticidal activity. Generally, the method involves planting a seed or seeds, infesting the resultant plant with insect nymphs at the appropriate growth stage to feed on the plant, maintaining the nymphs and plant in a sealed environment for at least as long as the nymph can survive without a food source, and evaluating the insect resistance of the plant.

Traditionally, plants are evaluated for insect resistance by selecting adult insects for use on mature plants or plant parts. However, the methods disclosed here exploit the observation that insects can be fed and sustained on emerging plants with fully expanded cotyledons. In specific embodiments, insects in the early developmental stages (i.e., second instar, third instar, etc.) can be fed and sustained on emerging plants in order to rapidly evaluate the insect resistance of the plant. Rather than waiting for mature plants and/or pods to develop, the methods disclosed herein provide the ability to screen and identify insect resistance (native or transgenic) with plants in the early developmental stages.

### A. Planting

As used in the method disclosed herein, any number of seeds can be planted for use in the method. For example, 1-10 seeds, 1-5 seeds, 1-3 seeds, 2-8 seeds, 2-5 seeds, 2-3 seeds, or 3-5 seeds can be planted in a single container for use in the method. Specifically, 1 seed, 2 seeds, 3 seeds, 4 seeds, 5 seeds, 6 seeds, 7 seeds, 8 seeds, 9 seeds, or 10 seeds can be planted in a single container for use in the method. Should multiple seeds be planted, emergent seedlings can be thinned to leave a single seedling for each container. For example, three seeds can be planted and, should three seedlings emerge from the soil, then two seedlings can be removed from the container such that only a single seedling is used in the remaining steps of the method disclosed herein. Similarly, multiple emergent seedlings can be thinned to leave 2 seedlings or 3 seedlings for use in the method described herein. In some embodiments, when one seedling emerges from the soil, another seed is planted such that two plants grow at different stages within the same container.

Seeds can be planted at any depth that allows for proper germination and plant development. For example, seed(s) for use in the method can be planted at a depth of about 0.1 in., 0.25 in., 0.3 in., 0.5 in., 0.75 in., 1.0 in., 1.25 in., 1.5 in., or any depth that allows proper germination and plant development. Likewise, soil employed for planting seeds for use in the method described herein can be any soil or growth medium commonly used to grow the plant of interest. The soil or growth medium should provide sufficient nutrients to allow for proper seed germination and plant development. Following planting, the soil or growth medium can be maintained in any environmental conditions (e.g., temperature, humidity, soil moisture content, hours of light, etc.)

sufficient to allow proper germination of the seed(s) and plant development. In order to maintain the soil or growth medium at the proper moisture content, water may be added from the top or bottom of the container. In some embodiments three soybean seeds are planted at a depth of 0.5 in.

Seeds for use in the method disclosed herein can be planted in any container capable of accommodating the proper amount of soil or growth medium, capable of allowing for plant development to the desired growth stage, capable of allowing for insect maintenance throughout the evaluation period, and capable of being sealed or enclosed, particularly once infested with an insect of interest. Further, containers may be designed with an enclosed space sufficient to allow infestation of the plant with insect nymphs and subsequent maintenance for proper evaluation of insect resistance as described elsewhere herein. Containers for use in the method described herein may be a single piece or multiple pieces. Containers may be appropriately scaled for larger or smaller quantities of plants and/or insects, as needed. Additionally, containers, or plant stakes within each container, can be marked for easy identification (e.g. bar coded) such that the method disclosed herein can be scaled up for high-throughput evaluation.

### B. Infesting

The method disclosed herein can be used to evaluate the resistance of any plant of interest to any insect of interest. In order to evaluate the insect resistance of a plant, the insect or insect nymph of interest can be infested on the plant of interest at any plant growth stage described elsewhere herein. As used herein, the term infesting refers to the combination of an insect with a plant or plant part. Infesting can occur when an insect is placed in close proximity to a plant of interest. For example, insects or insect nymphs can be infested onto a plant of interest at the emergence stage, during the growth stage, during the flowering stage, or during any pod production stage that the plant might have. Specifically, insect nymphs can be infested onto soybean plants at the VE stage, VC stage, V1 stage, V2 stage, or any other stage in which an insect nymph can utilize the plant as a food source. In some embodiments, insect nymphs are infested on a soybean plant at the VE stage, or the VC stage.

Likewise, the insect of interest can be infested on the plant of interest at any insect developmental stage as set forth elsewhere herein. For example, an insect nymph can be infested onto a plant of interest at the first instar, second instar, third instar, fourth instar, fifth instar, or during any developmental or adult growth stage. In certain embodiments, eggs can be infested onto a plant of interest or into an enclosed container having a planted seed that has not yet developed into a plant. In such embodiments, nymphs hatching from the infested eggs can feed on emergent plants following germination of the seed. When selecting nymphs for the method described herein, the appropriate growth stage of the nymph may be matched with the plant developmental stage such that plant material is available for the nymph to utilize as a food source. For example, when second instar nymphs are infested during the method disclosed herein, the second instar nymphs can be infested on plants at the emergence stage such that the second instar nymphs can use the emergent plant material as a food source. Similarly, when fourth instar or fifth instar nymphs are infested during the method disclosed herein, the fourth instar or fifth instar nymphs can be infested on mature plants or plant parts, such as mature soybean leaves or soybean pods, or soybean seedlings a the VC stage. In specific embodiments, brown marmorated stink bug nymphs or southern green stink bug nymphs at the second instar are infested on a soybean seedling at the VE, emergence stage.

Any number of insects of interest can be used for infesting in the method disclosed herein. For example, about 1-15, 1-10, 1-8, 1-7, 1-6, 1-5, 1-4, 1-3, 1-2, 2-10, 2-5, or 5-10 insects or insect nymphs can be used in the method disclosed herein. In some embodiments, at least about 1, about 2, about 3, about 4, about 5, about 6, about 7, about 8, about 9, about 10, about 12, about 15, or about 20 nymphs can be used for infesting in the method disclosed herein. In certain embodiments, at least about 5 southern green stink bug or at least about 5 brown marmorated stink bug nymphs at the second instar stage are infested onto soybean seedlings at the VE, emergence stage. In order to simultaneously determine insect resistance of a plant to multiple species of insects, more than one species of insect nymph can be infested onto a plant of interest. In certain embodiments, at least one brown marmorated stink bug nymph and at least one southern green stink bug nymphs are infested onto a soybean plant in order to determine resistance of soybean plant of interest to both brown marmorated stink bugs and southern green stink bugs.

Insects of interest can be infested onto a plant of interest by any means sufficient to maintain viability of the insect of interest and the plant of interest. In some embodiments an insect of interest is infested onto a plant of interest using a moistened camel hair brush such that the insect of interest is not injured and the plant of interest in not injured. Optionally, carbon dioxide gas can be used to knock down, stun, or anesthetize the insects prior to infesting. Alternatively, any other chemical or mechanical means can be used to knock down the insects prior to infesting. In some embodiments the environment around an infested plant is enclosed following infesting in order to keep insect nymphs in close proximity to the plant. As used herein, an enclosed environment refers to the immediate area around a plant or plant part such that insects of interest are in close contact with the plant of interest. The environment surrounding the plant can be enclosed by any means that maintains the insect of interest in close proximity to the plant of interest. In specific embodiments, five brown marmorated stink bug nymphs or five southern green stink bug nymphs at the second instar stage are infested on a soybean seedling at the VE, emergence, stage using a moistened camel hair brush.

### C. Feeding

The method disclosed herein can be useful in determining the insect resistance of a plant by maintaining an insect of interest in an enclosed and sealed environment with a plant of interest such that the insect has no food source other than the plant. In order to determine the insect resistance of a plant of interest, the insect of interest can be maintained on the plant for at least as many days as, or as long as, the insect can survive without food. By maintaining an insect of interest in a closed environment with a plant of interest for at least as many days as the insect can survive without food, the insect must use the plant as a food source in order to survive.

The number of days that an insect can survive without food can be determined by maintaining the insect in a sealed environment with water, but with no food source. Insects at different growth stages might be able to survive for different number of days without food. Thus, when determining the number of days that an insect can survive without food, the insect should be used at the same growth stage in which it would be infested onto a plant of interest. For example, if second instar insect nymphs are to be infested onto soybean plants, they should be maintained for at least as many days as a second instar nymph could survive on water without a food source. In some embodiments, insects of interest are maintained in an enclosed environment with a plant of interest for at least about 4-30 days, at least about 5-20 days, at least about 5-15 days, at least about 7-10 days, at least about 7-9 days, at least about 12-21 days, at least about 14-18 days, at least about 3 days, at least about 4 days, at least about 5 days, at least about 6 days, at least about 7 days, at least about 8 days, at least about 9 days, at least about 10 days, at least about 12 days, at least about 14 days, at least about 15 days, at least about 16 days, at least about 17 days, at least about 18 days, at least about 21 days, at least about 25 days, or at least about 30 days. The number of days the insects are maintained in the enclosed environment can be adjusted to account for changes in environmental conditions such temperature, hours of light, and humidity. In specific embodiments, a second instar brown marmorated stink bug nymph or a second instar southern green stink bug nymph is infested onto a soybean plant at the emergence stage and maintained in an enclosed environment for 7 days.

As used in the method disclosed herein, a plant of interest infested with an insect of interest should be maintained in conditions sufficient to sustain health of the plant. For example, water should be provided to the plant, from the top or the bottom of the plant or plant container, and the plant should be maintained in sufficient temperature, lighting, and humidity conditions for proper growth, development, and/or maintenance of the plant or plant part.

### D. Evaluating

The method disclosed herein can be useful for evaluating the insect resistance of a plant of interest. As used herein, the term “evaluating” is used to describe the process of assessing the resistance of a plant of interest to an insect. In one embodiment, insect resistance is evaluated by counting the number of stink bugs that survive longer than the number of days that the insect could survive without food. Any surviving insects would indicate that the insect can use the plant as a food source and, therefore, the plant of interest would be evaluated as not resistant, but would be sensitive, to the insect of interest. However, if the insects infested on the plant are not able to use the plant as a food source, the insects will not survive and the plant of interest would be evaluated as resistant to the insect of interest. After maintaining the plant and insect in an enclosed environment for at least as many days as the insect would survive without food, if more insects survive than do not survive, the plant may be evaluated as partially sensitive to the insect of interest. After maintaining the plant and insect in an enclosed environment for at least as many days as the insect would survive without food, if more insects do not survive than survive, the plant may be evaluated as partially resistant to the insect of interest.

Alternatively, in order to assess the resistance of a plant of interest to an insect of interest, the level of insect resistance of the plant of interest can be compared to a proper control plant. For example, the insect resistance of a soybean plant of interest to a southern green stink bug or brown marmorated stink bug can be compared to the insect resistance of resistant soybean variety IAC-100 (J Econ Entomol (2007) 100(3):962-8), or to any sensitive soybean plant, such as soybean variety XB28E07 (U.S. Pat. No. 7,485,778). If the same number, or about the same number, of nymphs survive after maintaining in an enclosed environment with the plant of interest as survive after being maintained in an enclosed environment with a resistant control plant, the plant of interest can be evaluated as resistant to the insect of interest.

In certain embodiments, insects feeding on the plant of interest will not survive as long as they would survive without a food source. In these circumstances, the plant can be evaluated as having insecticidal activity. As used herein, “insecticidal activity” of a plant refers to the ability of a plant, when consumed by an insect pest, to prevent the insect pest from surviving as long as the insect pest would have survived without having consumed the plant. Insecticidal activity of a plant is observed when an insect pest, having consumed a plant with insecticidal activity, does not survive as long as the insect would have survived without a food source.

Three general kinds of plant resistance to insects have been identified: antibiosis, antixenosis, and tolerance. Antibiosis (non-choice) is the plant's ability to reduce the survival, reproduction, and fecundity of the insect. Antixenosis (choice) is the plant's ability to deter the insect from feeding or identifying the plant as a food source. Tolerance is the plant's ability to withstand heavy infestation without significant yield loss. Thus, in certain embodiments, plants of interest can be evaluated as having antibiosis, antixenosis, or tolerance to an insect of interest.

In some embodiments, after evaluating a plant of interest for resistance to an insect of interest, the plant of interest can be selected for further evaluation. Following selection, the plant of interest can be evaluated using the method disclosed herein in duplicate, in triplicate, or in any number repetitions necessary to confirm the resistance of the plant of interest to the insect of interest.

A plant or plants selected by the method disclosed herein are also provided. The selected plants can be used in breeding to produce insect resistant lines, such as stink bug resistant soybean varieties or kudzu bug resistant soybean varieties. In some embodiments, the selected plants have been evaluated by performing the method disclosed herein a single time. In other embodiment a plant is selected after being evaluated using the method disclosed herein multiple times, such as 1 repetition, 2 repetitions, 3 repetitions, 4 repetitions, 5 repetitions, 6 repetitions, 7 repetitions, 8 repetitions, 9 repetitions, 10 repetitions, 12 repetitions, 15 repetitions, or 20 repetitions of the evaluation method disclosed herein. Further disclosed herein are seeds produced by a plant selected by the method disclosed herein.

The article “a” and “an” are used herein to refer to one or more than one (i.e., to at least one) of the grammatical object of the article. By way of example, “an element” means one or more elements.

All publications and patent applications mentioned in the specification are indicative of the level of those skilled in the art to which this disclosure pertains. All publications and patent applications are herein incorporated by reference to the same extent as if each individual publication or patent application was specifically and individually indicated to be incorporated by reference.

Although the foregoing invention has been described in some detail by way of illustration and example for purposes of clarity of understanding, it will be obvious that certain changes and modifications may be practiced within the scope of the appended claims.

### EXPERIMENTAL

### Example 1

### Southern Green Stink Bug Colony Maintenance

All life stages are maintained within a growth chamber. The temperature is maintained between 23-28° C. with 35% relative humidity. The light banks are set on a 16 light: 8 dark cycle.

A colony of southern green stink bugs (SGSB), Nezara viridula is reared in Sterilite® containers with dimensions: 23-⅝″L×16-⅜″W x 13-⅛″H. A six inch square is cut from the middle of the rearing container lid and a screen is placed over the hole and attached with hot glue. The containers are washed using a 10% bleach and soap mix and air dried. The bottoms and sides are coated with Rain X to deter stink bugs from escaping. Paper towels are taped to the bottom of each rearing container and changed weekly. Distilled water is used for washing to slow down the molding process.

The stink bugs are maintained on organic green beans, carrots, tomatoes, and snow peas and an assortment of raw organic nuts including almonds, peanuts, sunflower seeds, and walnuts. Groups of 50 to 80 adults are reared in each container. The adults mate and oviposit on Chem wipes placed within the rearing containers. The vegetables, nuts, and water source are replaced as needed. The cast skins and dead stink bugs are removed daily to maintain the colony health.

The egg masses are collected daily and placed in individual Petri dishes lined with 55 mm filter paper. All eggs are placed facing upward in the dishes. A moistened 2″ cotton dental wick is placed beside but not touching the egg mass. The wicks are checked daily and kept moist. When nymphs reach the second instar they are moved to Sterilite® containers and fed green beans and sunflower seeds.

### Example 2

### Brown Marmorated Stink Bug Colony Maintenance

All life stages are maintained within a small rearing room. The temperature is maintained between 22 to 28° C. with 45 to 60% relative humidity. The lights are set on a 16 light: 8 dark cycle.

A colony of brown marmorated stink bugs (BMSB), Halyomorpha halys is reared in BugDorms (12″×12″×12″) tents (MegaView Science Co., Ltd., Taichung, Taiwan). The stink bugs are maintained on Pioneer® variety XB28E07 soybean colony plants. They are given green beans, tomatoes, snow peas, carrots and organic raw nuts including almonds, walnuts, sunflower seeds and peanuts. Moistened dental wicks are placed in a 1 inch condiment cup and wetted with distilled bottled water. The adults oviposit on the underside of the leaves of the colony soybean plants. The plants are checked daily for egg masses and removed. Groups of 30 to 50 stink bugs are reared in each BugDorm.

The leaf material surrounding the egg masses is removed. The egg masses are placed in filter lined petri dishes with a moistened dental wick. The wick is checked daily and kept moist. Food is added when nymphs reach the second instar. The second instar nymphs are then moved to BugDorm tents and handled as little as possible. The food and water are replaced as needed.

### Example 3

### SGSB and BMSB Cotyledon Assay

A high throughput assay was designed to evaluate the soybean germplasm for resistance to southern green stink bug and brown marmorated stink bug. Insect screening containers are filled with soil and three soybean seeds are planted at a depth of ½ inch. A planting stake is placed within each bottom container with the genotype name and seed inventory barcoded. Each entry is replicated four times. The screening containers are placed in a black flat with holes. The containers are lightly watered over the top daily. The trays are placed within reach in a growth chamber (25° C./30% relative humidity). The seeds germinate for 5 to 7 days until reaching VE, emergence. The cups are thinned to two plants at the time of infestation.

Petri dishes containing newly molted second instars are removed from the growth chamber and used for infestation. Five second instar nymphs are selected using a moistened camel hair brush. The instars are gently added to the environment with the plant, and the environment is then enclosed. The screening containers are placed into a 48 position tray and then placed back into the growth chamber. The units are monitored daily and watered when needed from the bottom. After 7 days, the nymphal survival is recorded. Lines with little nymphal survival are repeated in the cotyledon assay with 8 reps to confirm resistance.

TABLE 1
Average nymph survival of southern green stink bug and
brown marmorated stink bug in a cotyledon assay.

| | Southern green | Brown marmorated |
|---|---|---|
| Genotype | stink bug† | stink bug‡ |
| Variety 41 | 0.7 | 0 |
| Variety 9A | 1.7 | 3.5 |
| XB28E07 | 4.1 | 4.8 |

†Fifteen reps of data
‡Ten reps of data

### Example 4

### SGSB Pod Assay

Resistant soybean plants at R6 (full seed) are infested with southern green stink bugs to determine the efficacy of the resistance on the adult stage. Two adult female stink bugs are isolated within each tube. The pod containers are constructed using ULINE clear tubes (3″×6″). Squares of organdy cloth are placed over one end using heavy duty rubber bands. A foam circle is fitted into one side; a slit is cut halfway through the foam circle. The foam is then slipped over the stem containing two to three pods. The foam circle is secured into the bottom of the tube. Two adults that molted from fifth instar that day are used for infesting. Synching the adult ages eliminates age related effects on mortality. The top is then secured with the organdy cloth and rubber band. The tube is stabilized to the main stem using strips of Velcro. The adult survival is monitored after 4 days, 7 days, and 14 days.

TABLE 2
Southern green stink bug adult survival from
4 to 14 days after infestation in pod assay.

| | | Days after infestation | | |
|---|---|---|---|---|
| Genotype | Pot | 4 | 10 | 14 |
| Variety 9A | 1 | 7† | 2 | 1 |
| Variety 9A | 2 | 9 | 0 | 0 |
| Variety 9A | 3 | 6 | 4 | 0 |
| 94Y82 | | 1 | 1 | 0 |

†Total number dead per pot. Each pot contained 5 reps.

TABLE 3
Total southern green stink bug adult survival
14 days after infestation in pod assay.

| | | Total dead after 14 days | |
|---|---|---|---|
| Genotype | Rep | Number | % |
| Variety 9A | 1 | 10† | 100 |
| Variety 9A | 2 | 9 | 90 |
| Variety 9A | 3 | 10 | 100 |
| 94Y82 | | 2 | 20 |

†Total number dead per pot. Each pot contained 5 reps.

### Example 4

### Kudzu Bug Colony Maintenance

All life stages are maintained within a growth chamber. The temperature is maintained between 23-28° C. with 35% relative humidity. The light banks are set on a 16 light: 8 dark cycle.

A colony of kudzu bugs, Megacopta cribraria are maintained on Pioneer® variety XB28E07 soybean colony plants. All life stages except 1st instars are reared in BugDorms (12″×12″×12″). The dead adults, cast skins, and dead plant material are removed weekly. The colony plants are replaced as needed.

The egg masses are collected twice a week and placed in individual Petri dishes lined with 55 mm filter paper. The eggs are oviposited on the underside of the leaves and on the sides of the BugDorm tents. The eggs are gently removed from the sides of the BugDorm and the plant material removed surrounding the egg masses. All eggs are placed facing upward in the dishes. A moistened 2″ cotton dental wick is placed beside the egg mass, not touching the egg mass. The wicks are checked daily and kept moist. When nymphs reach the 2nd instar they are moved to BugDorms with a single colony plant using a moistened camel hair paint brush. The BugDorms are cleaned out monthly using a 10% bleach solution and scrub brush.

### Example 5

### Kudzu Assay

A high throughput assay was designed to evaluate the soybean germplasm for resistance to kudzu bugs. Insect screening containers are filled with soil and three soybean seeds are planted at a depth of 0.5 inch. A planting stake is placed within each bottom container with the genotype name and seed inventory barcoded. Each entry is replicated four times. The screening containers are placed in a black flat with holes. The containers are lightly watered over the top daily. The trays are placed within reach in a growth chamber (25° C./30% relative humidity). The seeds germinate 10 to 12 days until reaching VC to V1. The cups are thinned to two plants at the time of infestation.

Colony plants containing fourth instar nymphs are removed from the BugDorms and used for infestation. Four fourth instar nymphs are selected using a moistened camel hair brush. The instars are gently placed on the unifoliates using a moistened camel hair paint brush. The lid of the screening container is snapped over the bottom unit containing the plants. The screening containers are placed into a 48 position tray and placed back into the growth chamber. The units are monitored daily and watered when needed from the bottom. After 14 days, the nymphal survival and plant health is recorded. Lines with little nymphal survival are repeated in the assay with 8 reps to confirm resistance.

1. A method of evaluating insect resistance in a plant comprising:
a) planting a seed and allowing said seed to germinate at least until plant emergence;
b) infesting said plant with at least one insect nymph;
c) maintaining said nymph in an enclosed environment for at least as many days as the nymph can survive without food; and
d) evaluating insect resistance of said plant.

2. The method of claim 1, wherein said nymphs are combined with said plant during the vegetative growth stage.

3. The method of claim 2, wherein said nymphs are combined with said plant at the emergence growth stage.

4. The method of claim 1, wherein said at least one insect nymph is added between the second instar stage and fifth instar stage.

5. The method of claim 4, wherein said at least one insect nymph is added at the second instar stage.

6. The method of claim 4, wherein said at least one insect nymph is added at the fourth instar stage.

7. The method of claim 1, wherein at least about 1-10 insect nymphs are combined with said plant.

8. The method of claim 7, wherein 5 insect nymphs are combined with said plant.

9. The method of claim 1, wherein said at least one insect nymph is maintained in said sealed area for at least about 7 days.

10. The method of claim 9, wherein said at least one insect nymph is maintained in said enclosed environment for at least about 7-10 days.

11. The method of claim 1, wherein said at least one insect nymph is maintained in said enclosed environment for at least about 14 days.

12. The method of claim 11, wherein said at least one insect nymph is maintained in said enclosed environment for at least about 14-18 days.

13. The method of claim 1, wherein said seed is planted in a container.

14. The method of claim 1, wherein 2-5 of said seeds are planted.

15. The method of claim 14, wherein 3 of said seeds are planted.

16. The method of claim 14 or 15, wherein said emergent plants are thinned to leave a single emergent plant.

17. The method of claim 1, wherein said seed is a dicot seed.

18. The method of claim 17, wherein said dicot seed is Brassica, sunflower, cotton, canola, safflower, tobacco, Arabidopsis, soybean, peach, or alfalfa.

19. The method of claim 18, wherein said seed is soybean seed.

20. The method of claim 1, wherein said seed is a monocot seed.

21. The method of claim 20, wherein said monocot is maize, wheat, rice, barley, sorghum, or rye.

22. The method of claim 1, wherein said at least one insect nymph is a Hemiptera nymph.

23. The method of claim 22, wherein said Hemiptera nymph is a Pentatomidae nymph.

24. The method of claim 23, wherein said Pentatomidae nymph is a green stink bug nymph, brown marmorated stink bug nymph, southern green stink bug nymph, rice stink bug nymph, or forest bug nymph.

25. The method of claim 24, wherein said at least one brown marmorated stink bug nymph or at least one southern green stink bug nymph.

26. The method of claim 1, wherein a soybean seed is planted and allowed to germinate until emergence, wherein 5 brown marmorated stink bug nymphs at the second instar stage or 5 southern green stink bug nymphs at the second instar stage are infested onto the soybean plant at the emergence stage, and wherein said brown marmorated stink bug nymphs or said southern green stink bug nymphs are maintained in an enclosed environment for 7 days with the soybean plant.

27. The method of claim 22, wherein said Hemiptera nymph is a kudzu bug nymph.

28. The method of claim 1, wherein a soybean seed is planted and allowed to reach the VC to V1 stage, wherein 5 kudzu bug nymphs at the fourth instar stage are infested onto the soybean plant at the VC to V1 stage, and wherein said kudzu bug nymphs are maintained in an enclosed environment for 14 days with the soybean plant.

29. The method of claim 1, wherein an insect resistant plant is selected after said evaluation.

30. A plant selected by the method of claim 29.

31. A seed produced by the selected plant of claim 30.
